(19) United States
(12) Patent Application Publication
(10) Pub. No.: US 2005/0112078 A1
(43) Pub. Date: May 26, 2005

(54) Plant-derived protein extract compositions and methods

(76) Inventors: Sekhar Boddupalli, San Jose, CA (US); Khalid Mahmood, Batavia, IL (US)

Correspondence Address: FOLEY & LARDNER LLP, 1530 PAGE MILL ROAD, PALO ALTO, CA, 94304, US

(21) Appl. No.: 10/988,186

(22) Filed: Nov. 12, 2004

Related U.S. Application Data
(60) Provisional application No. 60/520,556, filed on Nov. 13, 2003.

(51) Int. Cl. A61K 7/06; A61K 7/11; A61K 7/00; A61K 31/355; A61K 35/78

(52) U.S. Cl. 424/70.14; 424/401; 514/458; 424/757

(57) ABSTRACT

Disclosed are skin care compositions comprising a plant-derived protein extract, such as a soy protein extract or a colloidal oatmeal preparation, supplemented by an enrichment composition, such as a non-alpha tocopherol, and methods of making and using such compositions.

### CROSS-REFERENCE TO RELATED APPLICATION

This application claims the benefit of U.S. Provisional application Ser. No. 60/520,556 filed Nov. 13, 2003, which is incorporated herein in its entirety.

### FIELD OF THE INVENTION

This invention relates to plant-derived protein materials, especially certain materials derived from legumes, such as soybeans, or grains (grasses), such as oats, and associated skin care compositions, methods for use as cosmetic or therapeutic agents, and methods of preparation thereof.

### BACKGROUND OF THE INVENTION

Increasingly, consumers are turning to natural products as sources of ingredients for health and beauty enhancement. The success of the dietary supplement industry and the acceptance of and demand by mainstream consumers for “health foods” is testament to this phenomenon. Plant-derived materials are widely used to produce extracts, protein concentrates, and isolates, which are commonly used to produce cosmetic skin care compositions. Examples of currently available plant-derived cosmetic products include Aveeno® (colloidal oatmeal), SoySoft® (soy lipid-based skin creme), and others.

Legumes and certain grains are economical sources of protein-rich materials for cosmetics. Typically, such materials are extracted and concentrated, using aqueous or organic solvents, then subjected to additional treatments for formulation for consumer products. While preparation of such materials by extraction and concentration enhances the uniformity and handling properties of such materials, such procedures may also serve to deplete the materials of certain beneficial materials, such as certain oils and other phytonutrients.

By way of example, Avena sativa (oat) has been used extensively throughout history as a topical skin product in its “raw” ground form, dry, soaked or cooked. Extraction procedures have been refined so that extracts can be made from the entire oat, not just the grain. Colloidal oatmeal is produced by finely grinding the oat grain, and is used extensively in lotions, creams, shampoos, conditioners, soaps, ointments and the like, as well as in bath and cleansing products, and poultices. U.S. Pat. No. 6,368,579, incorporated herein by reference, describes a colloidal oatmeal composition for use in treating skin irritation and inflammation and for use as a sun block. Colloidal oatmeal is also used as a dermal cleanser or topical powder. In addition, oat-derived protein is considered to have beneficial effects for conditioning skin and hair.

Likewise, certain legumes (e.g., soy) are also used as sources of protein-based materials that are considered to be beneficial to skin. For example, PCT Publication WO 03/072079, incorporated herein by reference, describes the use of a soy extract for balancing so-called “combination” skin—facial skin consisting of both oily and dry skin regions. Similarly, U.S. Patent Publication U.S. 2003/0180339, incorporated herein by reference, describes the use of enzymatically hydrolyzed soy protein in skin care preparations.

In order to produce products that are acceptable to the average consumer, major producers have devised ways of processing plant protein source materials to produce extracts having desirable visual and tactile properties (e.g., uniformity, clarity, light-color); however, such processing steps may selectively, if inadvertently, remove certain desirable components of the raw material. Conversely, certain raw materials, namely soy, are now known to contain estrogenic materials, the so-called “phytoestrogens,” which are generally considered to be certain isoflavones (genistein and daidzein) that exhibit mammalian estrogen receptor binding activity. While some consumers may find such components to be desirable in their personal care products, for others, particularly infants and certain males, the presence of estrogenic activity may be undesirable.

Thus, it would be desirable to produce plant-derived protein compositions that are enriched or supplemented with certain beneficial components. It would be further desirable to have the option that such compositions be essentially free of estrogenic activities.

All publications, patents, and patent applications cited herein, whether supra or infra, are each hereby incorporated by reference in their entireties.

### SUMMARY OF THE INVENTION

In one aspect, this invention relates to skin care compositions useful for treating skin discomforts and inflammations, as well as maintaining and improving the appearance of normal skin. In another aspect, the invention relates to a method for ameliorating or reducing inflammatory conditions afflicting skin as well as, maintaining and improving the appearance normal skin. In yet another aspect, the invention relates to compositions for treating and preventing inflammatory conditions afflicting baby skin.

In one embodiment, the invention includes plant-derived protein extracts enriched with one or more additional components. In some instances, such components may be derived from the original plant source (endogenous), and may have been removed or depleted by processing; in other instances, the component may be from another source, preferably a naturally occurring source, such as another plant source (exogenous). Addition of such component(s) may replenish the beneficial effects of the original, un-processed material, or may preferably augment such effects, while providing a consumer-acceptable product for use in maintaining or improving the appearance of healthy skin or treating such skin maladies as irritation, inflammation, photosensitivity, photo-aging, sunburn, sun exposure, wound healing, irregular skin tone, irregular skin texture, treatment of erythema or redness, diaper rash, and the like.

In preferred embodiments, the cosmetic composition is formed from a plant-derived protein extract and an enriching supplement component. Commercially suitable plants include legumes and grasses (grains), but may be selected from any source, preferably a protein-rich source such as soy or oat. The enriching component may be endogenous to the original plant source or exogenous, as described above.

In an exemplary embodiment of the invention, the composition comprises a soy protein extract supplemented with between 1-98%, 10-80%, 20-70%, 30-60%, 40-50%, or at least 0.1%, 0.5%, 1%, 2%, 5%, or 10% (w/w) of a non-alpha tocopherol, such as delta-, beta- or gamma-tocopherol, or a non-alpha tocopherol enriched tocopherol composition. In one embodiment, the composition comprises greater than about 1% (w/w) of a non-alpha tocopherol or non-alpha tocopherol enriched tocopherol preparation. In another exemplary embodiment, the composition comprises a colloidal oatmeal preparation enriched as described above. One non-alpha tocopherol enriched tocopherol composition for use in the compositions of the invention is a beta-, gamma- and delta-tocopherol-enriched tocopherol composition. Other additions may include, in place of or in addition to the tocopherol component, other, preferably natural, components that confer and/or replenish, beneficial attributes to the extract. Exemplary additives include phytosterols and lecithins, as described herein.

According to another aspect, plant-derived protein extracts used in the compositions of the present invention will retain most of their native, beneficial properties. In a preferred embodiment, a soy protein extract will retain most of its protein function, as evidenced by at least about 80% of the trypsin inhibitory activity present in the starting material from which the soy protein extract is produced. Extraction conditions that facilitate such maintenance of protein function are described herein and are known in the art.

According to yet another aspect, plant-derived protein extracts of the present invention will be essentially depleted of mammalian estrogen receptor binding activity, as described herein. Generally, compositions of the present invention will be non-naturally occurring compositions and may be formulated to be administered topically, transdermally or orally.

In yet another embodiment, compositions of the present invention are suitable for treating baby skin, particularly in reducing irritation associated with inflammatory conditions that may develop in baby skin, such as diaper rash.

In still another embodiment, the invention includes a method of treating or ameliorating the symptoms of a skin condition in a mammalian subject, by administering to the subject (or having the subject self-administer) a cosmetically or therapeutically effective amount of any of the skin care compositions described above. Such administration is typically topical, but may also be nutritional or part of a dietary supplement regimen.

In a related embodiment, the invention includes a skin care kit, which includes any of the plant-derived protein extracts and enrichment material comprising a plant-derived extract composition as described above and instructions for applying such composition to the skin of a mammalian subject, preferably a human subject.

It is further understood that the components summarized above can be used in any combination, taking into consideration the basic teachings set forth herein.

These and other objects and features of the invention will become more fully apparent when the following detailed description is considered in conjunction with the Examples.

### DETAILED DESCRIPTION OF THE INVENTION

### I. Definitions

Unless otherwise stated, the following terms used in this Application, including the specification and claims, have the definitions given below. It must be noted that, as used in the specification and the appended claims, the singular forms “a”, “an,” and “the” include plural referents unless the context clearly dictates otherwise.

By “amelioration” is meant the prevention, reduction or palliation of a state, or improvement of the state of a subject; the amelioration of a stress is the counter-acting of the negative aspects of a stress. Amelioration includes, but does not require complete recovery or complete prevention of a stress.

A “body waste containment article” refers to any of a number of articles of clothing or articles designed to be worn next to the skin to absorb or contain body waste. Examples of body waste containment articles include, but are not limited to baby diapers, training pants, adult diapers, incontinence aids, sanitary napkins or pads, and the like.

“Colloidal oatmeal” is a powdered form of oats, resulting from the fine grinding (pulverizing) and further processing of whole grain oat. The term refers to the dry powder, or to the suspension in a liquid medium.

As used herein, the term “comprising” and its cognates are used in their inclusive sense; that is, equivalent to the term “including” and its corresponding cognates.

The term “cosmetics” includes make-up, foundation, and skin care products. The term “make-up” refers to products that leave color on the face, including foundations, mascara, concealers, eye liners, brow colors, eye shadows, blushers, lip colors, and so forth. The term “foundation” refers to liquid, cream, mousse, pancake, compact, concealer or like products that even out the overall coloring of the skin. Foundation is typically manufactured to work better over moisturized and/or oiled skin.

As used herein, “cosmetically-acceptable” means that the product(s), ingredients or compound(s) which the term describes are suitable for use in contact with tissues (e.g., the skin) without undue toxicity, incompatibility, instability, irritation, allergic response, or the like. This term is not intended to limit the ingredient/product to which it describes for use solely as a cosmetic product (e.g., the ingredient may be used as a prescription or over-the-counter pharmaceutical product).

As used herein, “cosmetically acceptable carrier” includes any and all solvents, dispersion media, coatings, antibacterial and antifungal agents, isotonic and absorption delaying agents and the like. The use of such media and agents for dermatologically active substances is well known in the art. Except insofar as any conventional media or agent is incompatible with the active ingredient, its use in the therapeutic compositions is contemplated. Supplementary active ingredients can also be incorporated into compositions of the invention.

By “essentially depleted of estrogen receptor binding activity” is meant a composition having less than about 10%, less than 5%, less than 2%, less than 1% or less than 0.1% of mammalian estrogen receptor binding activity of the source material when measured as a function of protein content of the source material, or as a function of another standard component of the source material or composition.

The term “inflammation” or “inflammatory condition” refers to a the state or symptoms of one or more of a number conditions as described herein, and generally characterized by elevation of one or more pro-inflammatory cytokines.

By “non-naturally-occurring composition” is meant a composition that is not found in this form in nature. A non-naturally-occurring composition can be derived from a naturally-occurring composition, e.g., as non-limiting examples, via purification, isolation, concentration, chemical modification (e.g., addition or removal of a chemical group), and/or, in the case of mixtures, addition or removal of ingredients or compounds. A non-naturally-occurring composition can comprise or be derived from a non-naturally-occurring combination of naturally-occurring compositions. Thus, a non-naturally-occurring composition can comprise a mixture of purified, isolated, modified and/or concentrated naturally-occurring compositions, and/or can comprise a mixture of naturally-occurring compositions in forms, concentrations, ratios and/or levels of purity not found in nature.

The term “pharmaceutical” refers to an agent or mixture of agents that is primarily intended to treat or ameliorate a disease or disorder. A pharmaceutical may be available only by prescription or may be available “over-the-counter” (OTC); in either case, its formulation and distribution are generally regulated by a governmental authority charged with such regulation, such as the Food and Drug Administration (FDA) in the United States.

The term “pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

The term “prodrug” means a pharmacologically inactive form of a compound which must be metabolized in vivo, e.g., by biological fluids or enzymes, by a subject after administration into a pharmacologically active form of the compound in order to produce the desired pharmacological effect. The prodrug can be metabolized before absorption, during absorption, after absorption, or at a specific site. Although metabolism occurs for many compounds primarily in the liver, almost all other tissues and organs, especially the lung, are able to carry out varying degrees of metabolism. Prodrug forms of compounds may be utilized, for example, to improve bioavailability, improve subject acceptability such as by masking or reducing unpleasant characteristics such as bitter taste or gastrointestinal irritability, alter solubility such as for intravenous use, provide for prolonged or sustained release or delivery, improve easy of formulation, or provide site-specific delivery of the compound. Reference to a compound herein includes prodrug forms of a compound.

The term “protein function” in the context of the present invention means that the proteins in the composition are not totally degraded and hence, inactive with regard to one or more of their recognized bioactivities, which may or may not be directly related to the intended role(s) or function(s) of the compositions of the invention. An exemplary measure of protein function is trypsin inhibitory activity exhibited by a trypsin inhibitor protein that is native to soy; other enzymatic or functional characteristics can also be used.

The term “retinoid” as used herein refers to retinoic acid, retinol, retinal and C2-C20 retinyl esters. Included in the term “retinoic acid” are 13-cis retinoic acid and all-trans retinoic acid. The term “retinol” includes the following isomers of retinol: all-trans-retinol, 13-cis-retinol, 11-cis-retinol, 9-cis-retinol, 3,4-didehydro-retinol. Preferred isomers are all-trans-retinol, 13-cis-retinol, 3,4-didehydro-retinol, 9-cis-retinol. Most preferred is all-trans-retinol, due to its wide commercial availability. Retinyl ester is an ester of retinol. Examples of retinyl esters include: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadeconoate, retinyl stearate, retinyl isostearate, retinyl nonadecanoate, retinyl arachidonate, retinyl behenate, retinyl linoleate, retinyl oleate, retinyl lactate, retinyl glycolate, retinyl hydroxy caprylate, retinyl hydroxy laurate, retinyl tartarate.

A “safe and effective amount” means an amount of compound or composition (e.g., the legume product) sufficient to induce a positive modification in the condition to be regulated or treated, but low enough to avoid serious side effects. The safe and effective amount of the compound or composition will vary with the particular condition being treated, the age and physical condition of the end user, the severity of the condition being treated/prevented, the duration of the treatment, the nature of other treatments, the specific compound or product/composition employed, the particular cosmetically-acceptable carrier utilized, and like factors.

The term “skin care composition” refers to a formulation that includes active ingredients, such as the compositions of the present invention, formulated for use in providing beneficial effects to the skin. Skin care compositions include, but are not limited to skin care products, pharmaceutical products and cosmetics, and may be formulated as topical, transdermal or oral compositions.

The term “skin care products” refers to products used to treat or otherwise care for, moisturize, improve the appearance or feel of, or clean the skin. Exemplary products covered by the phrase “skin care products” include, but are not limited to, adhesives, after-shave preparations, bandages, bath and shower products (soaps, gels, oils, bubble bath), toothpaste, anhydrous occlusive moisturizers, acne treatments, antiperspirants, clarifiers, deodorants, exfoliators, firming/cellulite treatments, hair care products (hairspray, shampoos, conditioners, hair gel, mousse, detanglers), lip products (moisturizers, balms and protectants), masks, oil/shine control, nail polish, powders, pore strips, self-tan products, shave preparations, skin lighteners, tissues, toners, wipes, solid emulsion compact, anhydrous hair conditioners, and the like.

The term “subject” or “individual” (used interchangeably herein) means mammals and non-mammals. A “mammal” may refer to any member of the class Mammalia. Examples of mammals include, but are not limited to: humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” or “individual” does not denote a particular age or sex.

The term “substantially pure” means at least about 90 mole percent, more preferably at least about 95 mole percent and most preferably at least about 98 mole percent of the desired enantiomer or stereoisomer is present compared to other possible configurations.

The term “therapeutically effective amount” means an amount of a compound that, when administered to a subject for treating a disease state, is sufficient to effect such treatment for the disease state. The “therapeutically effective amount” will vary depending on the compound, and disease state being treated, the severity or the disease treated, the age and relative health of the subject, the route and form of administration, the judgment of the attending medical or veterinary practitioner, and other factors.

By the term “tocopherol” is meant any of a family of molecules which are characterized by a 6-chromanol ring structure and a side chain at the 2 position. A “non-alpha tocopherol” is any of the tocopherols listed below, except alpha tocopherol. A “non-alpha-tocopherol enriched tocopherol composition”, as used herein refers to the non-alpha-tocopherol, such as for example, gamma-, beta- or delta-tocopherol as being enriched with respect to total tocopherols in the composition. Tocopherols possess a 4′,8′,12′-trimethyltridecyl phytol side chain. As used herein, the term “tocopherol” encompasses, but is not limited to:
alpha-tocopherol, [2R-2R*(4R*,8R*)]-3,4-dihydro-2,5,7,8-tetramethyl-2-(4,8,12-trimethyltridecyl)-2H-1-benzopyran-6-ol; 2,5,7,8-tetramethyl-2-(4′,8′,12′-trimethyltridecyl)-6-chromanol; 5,7,8-trimethyltocol, Fernholz (1937) J. Am. Chem. Soc. 59:1154 and 60:700;
beta-tocopherol, 3,4-dihydro-2,5,8-trimethyl-2-(4,8,12-trimethyltridecyl)-2H-1-benzopyran-6-ol; 2,5,8-trimethyl-2-(4,8,12-trimethyltridecyl)-6-chromanol; 5-8-dimethyltocol; cumotocopherol; neotocopherol; pxylotocopherol;
gamma-tocopherol, 3,4-dihydro-2,7,8-trimethyl-2-(4,8,12-trimethyltridecyl)-2H-1-benzyopyran-6-ol; 2,7,8-trimethyl-2-(4,8,12-trimethyltridecyl)-6-chromanol; 7,8-dimethyltocol; o-xylotocopherol;
delta-tocopherol, [2R-[2R*(4R*,8R*)]]-3,4-dihydro-2,8-dimethyl-2-(4,8,12-trimethyltridecyl)-2H-1-benzo-pyran-6-ol; 8-methyltocol;
epsilon-tocopherol, [R-(E,E)]-3,4-dihydro-2,5,8-trimethyl-2-(4,8,12-trimethyl-3,7,11-tridecatrienyl)-2H-1-benzopyran-6-ol; 2,5,8-trimethyl-2-(4,8,12-trimethyltrideca-3,7,11-trienyl)chroman-6-ol; 5-methyltocol;
zeta₁-tocopherol, 3,4-dihydro-2,5,7,8-tetramethyl-2-(4,8,12-trimethyl-3,7,11-tridecatrienyl)-2H-1-benzopyran-6-ol; 2,5,7,8-tetramethyl-2-(4,8,12-trimethyl-3,7,11-tridecatrienyl)-6-chromanol; 5,7,8-trimethyltocotrien-3′,7′,11′-ol;
zeta₂-tocopherol, 3,4-dihydro-2,5,7-trimethyl-2-(4,8,12-trimethyltridecyl)-2H-1-benzopyran-6-ol; 2,5,7-trimethyl-2-(4,8,12-trimethyltridecyl-6-chromanol; 5,7-dimethyltocol; and
eta-tocopherol, 3,4-dihydro-2,7-dimethyl-2-(4,8,12-trimethyltridecyl)-2H-1-benzopyran-6-ol; 2,7-dimethyl-2-(4,8,12-trimethyltridecyl)-6-chromanol; 7-methyltocol.

See The Merck Index (1996), Twelfth Edition, Merck & Co., Whitehouse Station, N.J., pp. 1620-1621 and 1712, and references cited therein. Other tocopherols include xi₁-, xi₂-, and sigma-tocopherols.

Generally speaking, commercially available dietary supplements of Vitamin E are alpha-tocopherol enriched compositions. As used herein, a “non-alpha-tocopherol enriched tocopherol composition” refers to a composition comprising at least 50% of any tocopherol except for alpha-tocopherol. In some examples, the non-alpha-tocopherol is gamma-tocopherol, or a metabolite thereof, beta-tocopherol, or a metabolite thereof, or delta-tocopherol or a metabolite thereof. A non-alpha tocopherol enriched tocopherol composition may comprise a mixture of tocopherols, including alpha-tocopherol, as long as the composition comprises at least 50% of a non-alpha tocopherol. As used herein, a “non-alpha-tocopherol metabolite” refers to a metabolite of a non-alpha-tocopherol, preferably a naturally occurring metabolite, such as for example, a gamma-tocopherol metabolite, such as 2,7,8-trimethyl-2-(beta-carboxyethyl)-6-hydroxychroman (gamma-CEHC); a beta-tocopherol metabolite, such as for example, beta-CEHC; or a delta-tocopherol metabolite, such as for example, delta-CEHC. In certain mammals (rats) given d-delta-tocopherol, a sulfate conjugate of 2,8-dimethyl-2-(2′-carboxyethyl)-6-chromanol was detected as a metabolite (Chiku, et al., J. Lipid Res., Vol 25, 40-48, 1984).

In the body of a subject, a non-alpha-tocopherol breaks down into metabolites. These are “naturally occurring metabolites.” The present invention encompasses the use of gamma-tocopherol enriched tocopherol compositions that further comprise a gamma-tocopherol metabolite such as gamma-CEHC, racemic gamma-CEHC and (S) gamma-CEHC. See for example, Wechter et al., U.S. Pat. No. 6,242,479 for disclosure of gamma-tocopherol metabolites, specifically incorporated herein by reference in its entirety. The present invention also encompasses the use of gamma-tocopherol metabolite enriched compositions that further comprise gamma-tocopherol.

The present invention encompasses the use of beta-tocopherol enriched tocopherol compositions that further comprise a beta-tocopherol metabolite such as 2,5,8-trimethyl-2-(2-carboxyethyl)-6-hydroxychroman (beta-CEHC). The present invention also encompasses the use of beta-tocopherol metabolite enriched compositions that further comprise beta-tocopherol.

The present invention encompasses the use of delta-tocopherol enriched tocopherol compositions that further comprise a delta-tocopherol metabolite such as 2,8-dimethyl-2-(2-carboxyethyl)-6-hydroxychroman (delta-CEHC). The present invention also encompasses the use of delta-tocopherol metabolite enriched compositions that further comprise delta-tocopherol.

The term “topical application” means directly laying on or spreading on outer skin using, e.g., by use of the hands or an applicator such as a wipe, puff, roller, or spray. As used herein, “topical carer” means one or more compatible solid or liquid filler diluents that are suitable for topical administration to a mammal. Examples of topical carriers include, but are not limited to, water, waxes, oils, emollients, emulsifiers, thickening agents, gelling agents, and mixtures thereof.

The term “transdermal application” generally refers to methods in which a composition is delivered selectively to one area or “patch” of skin by a special applicator that is designed to contact an area of the skin and continuously deliver compound to that are for a period time.

The term “treating” or “treatment” of a disease state includes:
1. preventing the disease state, i.e. causing the clinical symptoms of the disease state not to develop in a subject that may be exposed to or predisposed to the disease state, but does not yet experience or display symptoms of the disease state;
2. inhibiting the disease state, i.e., arresting the development of the disease state or its clinical symptoms; or
3. relieving the disease state, i.e., causing temporary or permanent regression of the disease state or its clinical symptoms.

The term “trypsin inhibitory activity” means the ability of the legume product at a concentration of 0.1% (w/w) to inhibit the activity of the protease trypsin, as measured by the assay set forth below in Example 2. In one embodiment, the legume products of the present invention have a trypsin inhibitory activity of at least about 15%. In a further embodiment, the legume products of the present invention have a trypsin inhibitory activity of at least about 25%, such as at least about 50%.

### II. Compositions of the Invention

The present invention generally includes compositions of a plant-derived protein product and an enriching agent. Such compositions may be used for any of a number of skin care purposes, including but not limited to providing benefits to healthy skin, for example, by improving its appearance. Compositions of the invention may also be used therapeutically for a variety of pharmaceutical conditions, such as inflammation, or to reduce the irritating effects of certain ingredients commonly used in skin products, such as retinoids. Specific utilities and methods of treatment are described in further detail in Section III herein.

This section will describe general embodiments compositions of the invention, with reference to specific examples. Generally, compositions of the invention include a plant-derived protein product enriched with a non-estrogen-binding component, such as a tocopherol or a phytosterol. Other components, such as lecithins, may also be added to enhance the formulation. While the compositions described below make reference to specific plant materials, it will be apparent to persons skilled in the art that the general principles described herein can be utilized to prepare compositions using a variety of plant-derived protein products.

### A. Plant-Derived Protein Extracts

This section describes certain exemplary plant-derived protein extracts useful in producing compositions of the invention. It is appreciated that plant-derived protein extracts can be made from a number of readily available plant sources, including, without limitation, legumes (e.g., alfalfa, soybeans, chickpeas, faba beans, lentils, smooth peas, pigeonpeas, mung beans), grasses (oats, wheat, barley), and other protein containing plant sources, using methods analogous to those described here or in accordance with methods known in the art.

### Soy Protein Sources

Soy extracts of the invention are generally prepared from a soy-derived protein product, generally either soy milk, or soy protein, derived from the soybean (e.g., Glycine max, Glycine soja, Glycine hispida, Soja hispida). The soy product may contain only a portion of the soybean (e.g., an extract of the soybean such as a lipid reduced soybean powder or filtered soymilk) or may contain the entire soybean (e.g., a ground powder of the legume). The soy product may be in the form of a fluid (e.g. soymilk) or a solid (e.g., a soybean powder or a soymilk powder or a curd). When in the form of a fluid, the term “soy product” refers to the solid constituents of the fluid that are derived from the soybean. Methods for producing soy products are well known in the art.

Soy product may also be made from soybean powder by grinding dry soybeans, and the soybean powder may then be lyophilized. Soy product can also be derived from soymilk or soymilk powder. Soymilk is a combination of solids derived from soybeans and water, the mixture of which has some or all of the insoluble constituents filtered off. Soymilk powder is evaporated soymilk, which in one embodiment, is in a lyophilized or spray-dried form.

Procedures for manufacturing soymilk include, but are not limited to, the following three procedures. First, soymilk may be made by placing soybeans into water to allow them to absorb the water. The swelled beans are then ground and additional water is then added. The mixture may then be filtered to remove any insoluble residue. Second, soymilk may also be prepared from soybean powder. Soybean powder is thoroughly mixed with water (e.g., for at least one hour), which may then be followed by a filtration process to remove insoluble residues. Third, soymilk can also be reconstituted from soymilk powder by adding water.

### Soy Protein Extracts

In accordance with the present invention, soy extracts are produced that retain, as much as possible, native protein function or activity. Protein function can be assessed by measuring one or more protein activities known to be present in the parent product, such as trypsin- or chymotrypsin- inhibitory activity, as described in the Examples herein. While a number of extraction procedures can be used, extracts used in the present invention may conveniently be derived from soymilk powder, such as can be commercially obtained. As detailed in Example 1 herein, approximately 5 grams of soymilk powder (Sunlight Foods Corp., Taipei, Taiwan, R.O.C.) is suspended in 100 mL of ethanol/methanol in water as a 50, 60, 80, or 100% solution. The mixture is extracted for 1 to 4 hours at 22° C. to 85° C., after which the mixtures are centrifuged and supernatants analyzed for isoflavone levels. The residue is washed three times with 100 mL of EtOH and water (80:20), then centrifuged and decanted. The residue is then lyophilized and analyzed by HPLC to measure isoflavone levels, and assayed for trypsin inhibition activity. Extracts produced in accordance with the present invention optimally retain 100%, and preferably at least about 75% percent of the trypsin inhibitory activity present in the parent product, though lesser activities may be acceptable for certain purposes.

Further, the foregoing extraction method has the advantage of removing at least about 70%, 80%, 90%, 95%, 98%, or 99% of mammalian estrogen receptor binding activity, as assessed in a standard estrogen receptor binding assay (recombinant estrogen receptors ER-alpha and ER-beta; MDS Pharma Services, Bothell, Wash.; www.mdsps.com). A soy extract that has less than 30%, less than 20%, less than about 10%, less than about 5%, or less than about 1% of the mammalian estrogen receptor binding activity of the parent plant material. Such extracts are said to be “essentially depleted of mammalian estrogen receptor binding activity.” Without ascribing to any particular theory, such estrogen receptor binding activity in plant materials is generally attributed to the presence of certain isoflavone compounds, such as daidzen, malonyl daidzen, genestin, malonyl genestin, and genestien. When extracts produced in accordance with the invention were assayed in parallel by HPLC, peaks comigrating with these compounds were significantly depleted in such extracts.

Other methodologies known in the art may be used to produce soy protein extracts suitable for use in the present invention. Suitability may be assessed by testing the protein function (i.e., positive trypsin inhibitory activity) and, preferably, lack of estrogenic activity (negative estrogen receptor binding), as described herein, or in accordance with standard methods known in the art.

### Oatmeal Protein Preparations

Colloidal oatmeal is produced from grinding and further processing of whole grain oat. Colloidal oatmeal and standard extracts thereof can be obtained from a number of commercial sources, including Nurture, Inc., Devon, Pa. Other oatmeal preparations include, for example, Nurture's Oat Protein™ (Nurture, Inc.), which is a defatted, undenatured oat protein in the form of fine microporous particles with native starch.

Although colloidal oatmeal is a cost-effective and efficacious ingredient, many of the beneficial ingredients present in the native oat, such as the oat beta-glucan, active oat extract and oat protein may be degraded or lost during the refining and separation process. The loss of these essential proteins and nutrients through refinement is thought to reduce significantly the beneficial properties of colloidal oatmeal, compared to the native plant.

Thus, a colloidal oatmeal based raw material enriched with an ingredient that would restore and/or augment its beneficial properties would be extremely desirable and acceptable to subjects who are experiencing the types of skin irritation or discomfort. Preparations containing such enriched compositions would also be beneficial to skin care maintenance for normal skin. Such compositions are included in the present invention.

### B. Enrichment of Plant-Derived Protein Extracts

It is a feature of the present invention that certain enriching materials, when added to plant derived protein extracts, provide additional benefits either lost during the refining process or not present or present at lower levels in the original plant protein product. This section describes certain enrichments that may be advantageously added to one or more of the plant-derived protein extracts described in the previous section, to obtain superior skin-beneficial properties to those exhibited by either the plant-derived protein extract or the enriching material alone.

U.S. Pat. No. 6,132,795, incorporated herein by reference, describes the preparation of a vegetable (soy) protein extract that is depleted of certain isoflavones and other ingredients, but to which isoflavones were added back, in order to take advantage of the known anti-inflammatory and other beneficial properties of these compounds; however, as mentioned above, such compounds have been found to have estrogenic activity, which is undesirable for many consumers. The present invention therefore provides a non-estrogenic alternative to such preparations.

### Non-Alpha Tocopherols

According to an important feature of the present invention, addition of one or more non-alpha-tocopherols, preferably gamma-, delta- or beta-tocopherol, to plant-derived protein extracts of the present invention confers additional anti-inflammatory properties, as well as certain skin photoprotective properties (sunscreen), and post-sun wound-healing properties (treatment of erythema), in addition to generally providing improved skin appearance and tone (reduced “blotchiness”), among others. According to an important feature of the invention, the beneficial effects of the combination of the plant-derived protein extract and the tocopherol are greater than would be predicted on the basis of the individual components.

As described in the Definitions section above, there is a variety of naturally occurring “non-alpha-tocopherols.” The predominant forms used in compositions of the present invention are beta-, gamma-, and delta-tocopherol, due, in part, to their relative prevalence in commonly occurring plants, such as corn and soybeans; however, it is appreciated that any of the non-alpha tocopherols can be substituted for these predominant forms, to practice the present invention. Non-alpha tocopherols can be obtained from known commercial sources. Cargill Health and Food Technologies (Minneapolis, Minn.), for example, produces a gamma-tocopherol enriched preparation (“Mixed Tocopherols”) comprising 50-70% gamma tocopherol, 15-30% delta tocopherol, <5% beta tocopherol, and <20% alpha tocopherol. This mixture is suitable for use in compositions of the present invention; more preferably, a gamma-tocopherol-enriched tocopherol preparation for use in compositions of the present invention will contain greater than 70%, greater than 80%, greater than 90% or greater than 95% tocopherol. Highly purified gamma tocopherol (>95%) can be obtained from Sigma Chemicals (St. Louis, Mo.). Delta-tocopherol is also produced commercially from cottonseed, maize, rice germ, soya been oil, wheat germ, or green leaves, according to methods known in the art, and can be obtained commercially, as well. Mixed tocopherol formulations including beta-, delta-, and gamma-tocopherols are sold as OTC dietary supplements; accordingly these ingredients may be used in oral, as well as topical and transdermal, formulations of the present invention.

Compositions of the present invention will optimally be formulated to contain at least 1%, more preferably 3%, and still more preferably, at least 5% of a non-alpha tocopherol or a non-alpha tocopherol enriched tocopherol composition, as defined above.

### Phytosterols and Phytosterol Esters

According to another embodiment, formulations of the present invention may include one or more phytosterols. Phytosterols are plant-derived lipids that may be removed during processing. A commercially available phytosterol preparation, is CoroWise™, manufactured by Cargill Health & Food Technologies (Minneapolis, Minn.) from plant sources, which contains 40-58% sitosterol, 20-28% campesterol, and 14-23% stigmasterol. Based on data from scores of trials conducted on the use of phytosterols in the diet, a daily intake of at least 0.8 grams of free phytosterols and/or 1.3 grams of phytosterol esters (e.g., CoroWise physterol esters Cargill), as a part of a diet low in saturated fat and cholesterol has been recommended to provide significant cholesterol lowering benefits. Accordingly, this ingredient may be used in oral, as well as topical and transdermal, formulations of the invention.

### Other Additives

Formulations of the present invention may be further augmented with formulation additives, such as a number of compounds generally used in formulating cosmetic or pharmaceutical products, as are well known in the art. In particular, formulations of the invention may be further improved by addition of lecithins, which may be removed during processing, such as during preparation of the soy extracts described in conjunction with practice of the present invention. Lecithins are comprised predominantly of phospholipids, and are natural surfactant compounds found in soybeans, rice and other plant materials. One suitable commercially available lecithin preparation is Lecigran®, sold by Riceland Technologies, Inc. (Stuttgart, Ak., USA) which contains the following approximate components: phosphatidylcholine, 26%; choline, 3%; phosphatidylethanolamine, 20%; inositol phosphatides, 14%; inositol, 2.2%; phosphatidylserine, <1%; phytoglycolipids, 13%; other phosphatides, lipids, 14.5%; soybean oil, 2%. This ingredient is also suitable for oral, as well as topical and transdermal formulations of the present invention.

Other formulatory components will be known and available to skilled practitioners in the art of cosmetic formulations, as described in further detail below.

### III. Utility; Methods of Use

Compositions in accordance with the present invention may be used either alone or as part of topical and transdermal formulations for a number indications, such as symptoms of inflammation and skin irritation, and maintaining or improving the appearance of healthy tone, color and body of skin, as further described in Part A of this section. Since, for the most part, the ingredients described herein are either known dietary supplements, known to be consumed by humans in standard diets, and/or generally recognized as safe (GRAS) by the U.S. Food and Drug Administration (FDA), they may additionally be formulated as oral preparations (i.e., nutritional or dietary supplements) for these purposes, subject to the regulatory authorities of the countries in which they are intended to be used.

Topical agents in accordance with the present invention (creams, ointments, liniments and the like), as well as shampoos, conditioners and bath products may be utilized for treating skin discomforts as well as for maintaining normal skin. Formulations of varying ointments, creams, aqueous solutions, liniments, shampoos, conditioners, bath products and the like for treating skin discomforts as well as improving the appearance of skin are known, and are described in further detail in Section IV herein.

### A. Indications

Generally, formulations of compositions of the invention may be used to provide anti-inflammatory, anti-oxidative cellular stress, anti-acne, sunscreen, post-sun photo repair, post-sun wound healing, relief from erythema or redness (“sunburn”), improvement of skin tone and texture, skin lightening (de-pigmentation), and reduction of retinoid-induced irritation. Assessment of efficacy of a particular formulation may be made in one or more pre-clinical or clinical assays known in the art, including, but not limited to: E-selectin cellular assay (anti-inflammation), Glutathione depletion assay (oxidative stress), De-pigmentation assays (cellular epidermal cells, inhibition of pigmentation in dark skinned microswine). Appropriate pre-clinical assays are detailed in the Examples section.

1. Skin Inflammation. Compounds and methods of the invention may be employed in any skin care application where decreased inflammatory response is desirable. For example, compounds and compositions of the invention may be incorporated into leave-on and rinse-off acne preparations, facial milks and conditioners, shower gels, foaming and non-foaming facial cleansers, cosmetics, hand and body lotions, leave-on moisturizers, cosmetic and cleaning wipes, salves for poison ivy, chicken pox, or pruritis, or the like. Generally, for dermal applications, topical administration is preferred; however, systemic administration, particularly oral administration, as described elsewhere herein, is also possible.

2. Retinoid-induced Skin Irritation. Retinoids have been shown to enhance keratinocyte proliferation in vitro, increase epidermal thickness and increase collagen synthesis by dermal fibroblasts. These attributes have lead to inclusion of retinoids in formulations used, for example, for protection from sun damage and smoothening of wrinkled skin.

A drawback of retinoids is their tendency to cause skin irritation, usually presenting as mild erythema and stratum corneum peeling of the skin. It has been suggested that the pro-inflammatory cytokines interleukin-1 (IL-1) and monocyte chemoattractant protein I (MCP-1) may serve as mediators in such retinoid-induced dermatitis (Kim et al., 2003, Toxicol. Lett. 146: 65-73).

Formulations of the present invention may be screened in vitro by testing ability to reduce secretion of MCP-1 and IL-8 in cultured fibroblasts, as described by Kim et al. Reagents for detecting these cytokines are commercially available, for example, from Cell Sciences, Canton, Mass., USA. In vivo efficacy tests for the reduction of retinol-induced irritancy can be performed using a standard human patch test.

In accordance with one embodiment, formulations of the invention can be added to retinol-containing products or can be administered, either topically or orally, in conjunction with such products, to reduce retinol-induced skin irritation.

3. Sunburn. Exposure to sunlight can result in damage to skin, particularly light-colored skin. The major short-term hazard of prolonged exposure to sunlight is erythema, i.e., sunburn, which primarily results from UVB radiation having a wavelength of from about 290 nm to about 320 nm. Over the long term, however, such prolonged exposure can often cause malignant changes in the skin. Epidemiologic studies have shown a strong relationship between sunlight exposure and human skin cancer.

Another long-term hazard of ultraviolet radiation is premature aging of the skin, which is primarily caused by UVA radiation having a wavelength of from about 320 nm to about 400 nm. This condition is described in further detail in Sub-part 4 of this section, below.

The compositions of the present invention are suitable for providing protection against the harmful effects of ultraviolet radiation. Compositions of the present invention are suitable for use in sunscreen preparations to provide protection to human skin from the harmful effects of UV radiation, which include, but are not limited to, sunburn and premature aging of the skin. While the first line of defense against the harmful effects of UV radiation generally involve attenuating or reducing the amount of UV radiation that reaches the skin's surface, additional protection and benefit can be provided by attenuating inflammatory reactions to such UV and/or treating sunburn. Accordingly, the methods of treatment for the harmful effects of ultraviolet radiation also include administration of a composition of the invention after the exposure to UV radiation has already taken place.

4. Skin Appearance and Aging. The compositions of the present invention are also useful for regulating the condition of or improving the appearance of the skin, including visible and/or tactile discontinuities in skin. Such discontinuities may be induced or caused by internal and/or external factors, and include the signs of skin aging described herein.

As mentioned above, premature skin aging is primarily caused by UVA radiation having a wavelength of from about 320 to about 400 nm. This condition is characterized by wrinkling and pigment changes of the skin, along with other physical changes such as cracking, telangiectasis, solar dermatoses, ecchymoses, and loss of elasticity. Individuals, particularly those having light-skin who burn easily and tan poorly, who have had a great deal sun exposure in childhood can show the following gross cutaneous alterations in later adult life: wrinkling, leatheriness, yellowing, looseness, roughness, dryness, mottling (hyperpigmentation) and various premalignant growths (often subclinical). These cumulative effects of sunlight are often referred to as “photoaging”. Although the anatomical degradation of the skin is most advanced in the elderly, the destructive effects of excessive sun exposure are already evident by the second decade. Serious microscopic alterations of the epidermis and dermis occur decades before these become clinically visible. Wrinkling, yellowing, leatheriness and loss of elasticity are very late changes.

Signs of skin aging may also be induced or caused by other physiological and environmental stimuli (i.e., smoke, ozone, pollutants, stress, etc.). These signs may result from processes which include, but are not limited to, the development of textural discontinuities such as wrinkles, including both fine superficial wrinkles and coarse deep wrinkles, skin lines, facial frown lines, expression lines, rhytides, dermatoheliosis, photodamage, premature skin aging, crevices, bumps, pits, large pores (e.g., associated with adnexal structures such as sweat gland ducts, sebaceous glands, or hair follicles), “orange-peel” skin appearance, dryness, scaliness, flakiness and/or other forms of skin unevenness or roughness; blemishes such as acne, pimples, breakouts; excess skin oil problems such as over production of sebum, oiliness, facial shine, foundation breakthrough; abnormal desquamation (or exfoliation) or abnormal epidermal differentiation (e.g., abnormal skin turnover) such as scaliness, flakiness, keratoses, hyperkeratinization; inadequate skin moisturization (or hydration) such as caused by skin barrier damage, environmental dryness; loss of skin elasticity (loss and/or inactivation of functional skin elastin) such as elastosis, sagging (including puffiness in the eye area and jowls), loss of skin firmness, loss of skin tightness, loss of skin recoil from deformation; non-melanin skin discoloration such as under eye circles, blotching (e.g., uneven red coloration due to, e.g., rosacea), sallowness (pale color), discoloration caused by telangiectasia or spider vessels; melanin-related hyperpigmented (or unevenly pigmented) skin regions such as age spots (liver spots, brown spots) and freckles; post-inflammatory hyperpigmentation such as that which occurs following an inflammatory event (e.g., as an acne lesion, in-grown hair, insect/spider bite or sting, scratch, cut, wound, abrasion, and the like); atrophy such as, but not limited to, that associated with aging or steroid use; other histological or microscopic alterations in skin components such as ground substance (e.g., hyaluronic acid, glycosaminoglycans, etc.), collagen breakdown and structural alterations or abnormalities (e.g., changes in the stratum corneum, dermis, epidermis, the skin vascular system such as telangiectasia or spider vessels); tissue responses to insult such as itch or pruritus; and alterations to underlying tissues (e.g., subcutaneous fat, cellulite, muscles, trabeculae, septae, and the like), especially those proximate to the skin.

Compositions of the invention may regulate or reduce the signs of skin aging by prophylactically regulating and/or therapeutically regulating one or more of such signs (similarly, regulating a given sign of skin aging, e.g., lines, wrinkles or pores, includes prophylactically regulating and/or therapeutically regulating that sign). As used herein, prophylactically regulating such signs includes delaying, minimizing and/or preventing signs of skin aging. As used herein, therapeutically regulating such signs includes ameliorating, e.g., diminishing, minimizing and/or effacing signs of skin aging. Topical or oral formulations may be used for these purposes.

5. Cosmetic and Skin Care Products. Compositions of the present invention may also be used in cosmetic compositions and skin care products. Cosmetic compositions of the present invention are ideally suited for use in treating the skin and lips, especially in the form of a lipstick or lip balm for applying to the lips a permanent or semi-permanent color, optionally with a gloss or luster finish. The cosmetic compositions can also be used in treating the skin and/or lips with a skin care agent for protection against exposure to adverse weather, including the wind and rain, dry and/or hot environments, environmental pollutants (e.g., ozone, smoke, and the like), or exposure to excessive doses of sunlight, as described in a previous section. The compositions are also useful in preparations having as their primary goal moisturizing and/or conditioning for the hair and skin, improved skin feel, regulating skin texture, reducing fine lines and wrinkles, skin lightening, or the like, by inter alia, buffering or reducing the inflammatory effects of certain pro-inflammatory ingredients contained therein, for example, retinol.

The compositions of the invention can accordingly be applied to the skin and/or lips in the traditional manner with or without a conventional holder or applicator to provide a decorative and/or protective film thereto. Cosmetics include make-up, such as foundations, mascara, concealers, eye liners, brow colors, eye shadows, blushers, lip colors, and so forth.

Skin care products are products that are used to treat or otherwise care for, moisturize, improve the appearance or feel of, or clean the skin. Skin care products include, but are not limited to, adhesives, acne-care, after-shave preparations, bandages, bath and shower products (soaps, gels, oils, bubble bath), toothpaste, anhydrous occlusive moisturizers, acne treatments, antiperspirants, clarifiers, deodorants, exfoliators, firming/cellulite treatments, hair care products (hairspray, shampoos, conditioners, hair gel, mousse, detanglers), lip products (moisturizers, balms and protectants), masks, oil/shine control, nail polish, powders, pore strips, self-tan products, shave preparations, skin lighteners, tissues, toners, wipes, solid emulsion compact, anhydrous hair conditioners, and the like.

By way of further example, compositions and methods of the present invention may be useful in treating acne, a skin condition characterized by a profound inflammatory component.

6. Baby Skin Care. It is well known that the feel and character of baby skin is dramatically different from that of adult skin. These differences are due to more than relative lack of exposure to sunlight and other environmental insults. For example, the relative vulnerability in pre-term infants of the upper epidermal portion of the skin (stratum corneum), which provides a primary barrier to infection and environmental insults, including percutaneous drug absorption, has been the focus of much study (Mancini, 2004, Pediatrics 113: 1114-1119). The underlying dermis has not been as well studied; however, it is known that it is less well developed than that of adults. For example, connective tissue continues to accumulate throughout infancy. Somewhat surprisingly, damage to newborn skin tissue can lead to extensive scarring, particularly when the basal cell layer, which generates the epidermis, is damaged, as during surgery (Rutter, 2000, Semin. Neonatol., 5: 297-302).

Accordingly, compositions of the present invention, particularly those that are depleted of phytoestrogens, are useful in compositions that are directed at healing, soothing, relieving inflammation and irritation in baby skin.

Baby skin conditions that may benefit from the methods of the present invention include, but are not limited to, diaper rash, a common form of contact dermatitis and irritation occurring in infants, as well as adults, who wear diapers. U.S. Pat. No. 6,211,186, incorporated herein by reference, describes possible etiologies and methods of treating this condition. It is generally thought that one or more fecal and lipolytic enzymes, as well as ammonia, bacteria, urine pH, overhydration and Candida albicans may be involved in the onset of skin irritation and inflammation associated with diaper rash. It is also likely that physiological responses of the skin to the irritants, such as production of cytokines by keratinocytes, contribute to the ensuing appearance of erythema, papules, scaling and ulceration characteristic of the condition.

Disposable diapers are increasingly popular for containing waste from babies, as well as incontinent adults. These products have a high capacity for absorbing urine and other body exudates. They generally comprise some sort of liquid-pervious topsheet material, an absorbent core, and a liquid-impervious backsheet material. Although these types of absorbent structures may be highly efficient for the absorption of liquids, it is well recognized that long-term wear of such structures may lead to skin which is compromised in terms of being over hydrated or exposed to skin irritants commonly found in body exudates. It is generally known that skin under absorbent articles is more susceptible to skin disorders, including diaper rash, erythema (i.e., redness), heat rash, abrasion, pressure marks and skin barrier loss. Diaper rash is further characterized as an inflammatory condition caused by one or more of the following factors: moisture, occlusion, chafing, continued contact with urine or feces or both, or mechanical or chemical irritation.

A common treatment for diaper rash is application of a soothing ointment to the affected area. Typically such ointments contain zinc oxide as an active ingredient. Compositions of the present invention can be formulated to treat diaper rash in the form of a cream or ointment; alternatively, or in addition compositions of the present invention can be used in conjunction with currently available ointments, such as zinc-oxide based ointments, to provide adjunct anti-inflammatory activity.

Alternatively, or in addition, compositions of the present invention may be formulated to be delivered directly to the site of diaper-induced inflammation. U.S. Pat. No. 6,803,496, incorporated herein by reference, describes specific ways of impregnating fibrous materials, such as the topsheet portion of a diaper, with protective or therapeutic compositions, such as those of the present invention. It is further understood that compositions of the present invention may be used in a number of body waste containment articles, including but not limited to baby diapers, training pants, adult diapers, adult incontinence aids, sanitary napkins, and the like.

Due to shelf-life considerations, the formulations that are useful in this embodiment of the invention have a melting profile such that they are relatively immobile and localized on the wearer-contacting surface of the diaper at room temperature, are readily transferable to the wearer at body temperature, and yet are not completely liquid under extreme storage conditions. Preferably, the compositions are easily transferable to the skin by way of normal contact, wearer motion, and/or body heat.

In one embodiment, the diaper-immobilized skin care compositions are solid, or more often semi-solid, at 20° C., i.e. at ambient temperatures. By “semisolid” is meant that the composition has a rheology typical of pseudoplastic or plastic liquids. When no shear is applied, the compositions can have the appearance of a semi-solid but can be made to flow as the shear rate is increased. This is due to the fact that, while the composition contains primarily solid components, it also includes some minor liquid components. Preferably, the compositions of the present invention have a zero shear viscosity between about 1.0×10⁶ and 1.0×10⁸ centipoise or between 5.0×10⁶ and 5.0×10⁷ centipoise, where the term “zero shear viscosity” refers to a viscosity measured at very low shear rates (e.g., 1.0 sec⁻¹) using plate and cone viscometer (a suitable instrument is available form TA Instruments of New Castle, Del. as model number CSL 100). One of skill in the art will recognize means other than high melting point components (as discussed below) can be used to provide comparable viscosities measured for such compositions comprising such means can be measured by extrapolating a plot of viscosity vs. shear rate for such compositions to a shear rate of zero at a temperature of about 20° C.

For compositions designed to provide a therapeutic and/or skin protective benefit, a useful active ingredient in these compositions is one or more skin protectants or emollients. As used herein, the term “emollient” is a material that protects against wetness or irritation, softens, soothes, supples, coats, lubricates, moisturizes, protects and/or cleanses the skin. Representative emollients are discussed in the next Section IV; in the context of the immobilized diaper rash product discussed above, it will appreciated that emollients having “waxier” compositions at room temperature, such as petrolatum, may be more suitable than more fluid compositions.

Another optional, but useful component of the therapeutic/skin protective compositions described herein is an agent capable of immobilizing the composition (including the preferred emollient and/or other skin condition/protective agents) in the desired location in or on the treated article (i.e., the diaper). Because certain of the preferred emollients in the composition have a plastic or liquid consistency at 20° C., they tend to flow or migrate, even when subjected to modest shear. When applied to a wearer-contacting surface or other location of an absorbent article, especially in a melted or molten state, the emollient will not remain primarily in or on the treated region. Instead, the emollient will tend to migrate and flow to undesired regions of the article.

Specifically, if the emollient migrates into the interior of the article, it can cause undesired effects on the absorbency of the article core due to the hydrophobic characteristics of many of the emollients and other skin conditioning agents used in the compositions useful in the methods of the present invention. It also means that much more emollient has to be applied to the article to get the desired therapeutic and/or protective benefits. Increasing the level of emollient not only increases the cost, but also exacerbates the undesirable effect on the absorbency of the article's core and undesired transfer of composition during processing/converting of the treated articles.

The immobilizing agent counteracts this tendency of the emollient to migrate or flow by keeping the emollient primarily localized on the surface or in the region of the article to which the composition is applied. This is believed to be due, in part, to the fact that the immobilizing agent raises the melting point and/or viscosity of the composition above that of the emollient. Since the immobilizing agent is preferably miscible with the emollient (or solubilized in the emollient with the aid of an appropriate emulsifier), it entraps the emollient on the surface of the article's wearer contacting surface or in the region to which it is applied.

The immobilizing agent counteracts this tendency of the emollient to migrate or flow by keeping the emollient primarily localized on the surface or in the region of the article to which the composition is applied. This is believed to be due, in part, to the fact that the immobilizing agent raises the melting point and/or viscosity of the composition above that of the emollient. Since the immobilizing agent is preferably miscible with the emollient (or solubilized in the emollient with the aid of an appropriate emulsifier or dispersed therein), it entraps the emollient on the surface of the article's wearer contacting surface or in the region to which it is applied.

It is also advantageous to “lock” the immobilizing agent on the wearer contacting surface or the region of the article to which it is applied. This can be accomplished by using immobilizing agents which quickly set up (i.e., solidify) upon application to the article. In addition, outside cooling of the treated article via blowers, fans, cold rolls, etc. can speed up crystallization of the immobilizing agent.

In addition to being miscible with (or solubilized in) the emollient, the immobilizing agent will preferably have a melting profile that will provide a composition that is solid or semisolid at ambient temperature. In this regard, preferred immobilizing agents will have a melting point of at least about 35° C. This is so the immobilizing agent itself will not have a tendency to migrate or flow. Preferred immobilizing agents will have melting points of at least about 40° C. Typically, the immobilizing agent will have a melting point in the range of from about 50° to about 150° C.

When utilized, immobilizing agents useful herein can be selected from any of a number of agents, so long as the preferred properties of the skin care composition provide the skin benefits described herein. Preferred immobilizing agents will comprise a member selected from the group consisting of C14 -C22 fatty alcohols, C12-C22 fatty acids, and C12-C22 fatty alcohol ethoxylates having an average degree of ethoxylation ranging from 2 to about 30, and mixtures thereof. Additional immobilizing agents include C16-C18 fatty alcohols, most preferably crystalline high melting materials selected from the group consisting of cetyl alcohol, stearyl alcohol, behenyl alcohol, and mixtures thereof. (The linear structure of these materials can speed up solidification on the treated absorbent article.) Mixtures of cetyl alcohol and stearyl alcohol are particularly preferred. Other preferred immobilizing agents include C16-C18 fatty acids, most preferably selected from the group consisting of palmitic acid, stearic acid, and mixtures thereof. Mixtures of palmitic acid and stearic acid are particularly preferred.

Published U.S. Patent application publication number 2002/0106388, incorporated herein by reference, describes formulations in a microencapsulated form in a slurry, which is used to impregnate body garments, such as pantyhose, for extended application to the user's skin. Such application may be advantageous in conjunction with the compositions of the present invention, particularly as they refer to diapers or other absorbent articles described above.

### IV. Administration and Formulations

The present invention includes cosmetic and pharmaceutical compositions comprising a plant-derived protein extract in conjunction with at least one or more enrichment agents, for example, a non-alpha tocopherol. Such compositions will be optionally combined with at least one cosmetically- or pharmaceutically acceptable carrier, and optionally other beneficial ingredients, such as, for example, phytosterols, and/or lecithin, as described above.

In general, the compounds of the present invention will be administered in a therapeutically- or cosmetically-effective amount by any of the accepted modes of administration for agents that serve similar utilities. For cosmetic uses, formulations will be made to suit usual and customary rates of application by the ordinary consumer. For oral administration, suitable dosage ranges are typically 1-1000 mg daily, preferably 1-800 mg daily, and most preferably 1-500 mg daily, depending upon numerous factors such as the age and relative health of the subject, the potency of the formulation used, and the indication towards which the administration is directed, and the preferences and experience of the consumer involved. One of ordinary skill in the art of cosmetic or therapeutic formulations will be able, without undue experimentation and in reliance upon personal knowledge and the disclosure of this Application, to ascertain a therapeutically effective amount of the compounds of the present invention for a given indication.

As used herein, “cosmetically acceptable carrier” or “pharmaceutically acceptable carrier” includes any and all solvents, dispersion media, coatings, antibacterial and antifungal agents, isotonic and absorption delaying agents and the like. The use of such media and agents for dermatologically active substances is well known in the art. Except insofar as any conventional media or agent is incompatible with the active ingredient, its use in the therapeutic compositions is contemplated. Supplementary active ingredients can also be incorporated into the compositions.

For topical administration, the subject compositions may be provided as a wide variety of product types including, but are not limited to, lotions, creams, gels, sticks, sprays, mousses, emollients, ointments and pastes. These product types may comprise several types of formulations including, but not limited to solutions, emulsions, gels, solids, and liposomes.

Compositions useful for topical administration of the compositions of the present invention formulated as solutions typically include a cosmetically- or pharmaceutically-acceptable aqueous or organic solvent. The terms “cosmetically-acceptable organic solvent” and “pharmaceutically-acceptable organic solvent” refer to a solvent which is capable of having a composition of the present invention dispersed or dissolved therein, and of possessing acceptable safety properties (e.g., irritation and sensitization characteristics). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, isopropanol, butanetriol, sorbitol esters, 1,2,6-hexanetriol, ethanol, isopropanol, butanediol, and mixtures thereof.

If the topical compositions useful in the subject invention are formulated as an aerosol and applied to the skin as a spray-on, a propellant may be added to a solution composition. Examples of propellants useful herein include, but are not limited to, the chlorinated, fluorinated an chloro-fluorinated lower molecular weight hydrocarbons, such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin, described above.

Topical compositions useful in the subject invention may be formulated as a solution comprising an emollient. As used herein, “emollients” refer to materials used for the prevention or relief of dryness, as well as for the protection of the skin. A wide variety of suitable emollients is known and may be used herein. Representative emollients useful in the present invention include, but are not limited to, emollients that are petroleum-based; sucrose ester fatty acids; polyethylene glycol and derivatives thereof; humectants; fatty acid ester type; alkyl ethoxylate type; fatty acid ester ethoxylates; fatty alcohol type; polysiloxane type; propylene glycol and derivatives thereof; glycerine and derivatives thereof, including glyceride, acetoglycerides, and ethoxylated glycerides of C12-C28 fatty acids; triethylene glycol and derivatives thereof; spermaceti or other waxes; fatty acids; fatty alcohol ethers, particularly those having from 12 to 28 carbon atoms in their fatty chain, such as stearic acid; propoxylated fatty alcohols; other fatty esters of polyhydroxy alcohols; lanolin and its derivatives; kaolin and its derivatives; any of the monographed skin care agents listed above; or mixtures of these emollients. Suitable petroleum-based emollients include those hydrocarbons, or mixtures of hydrocarbons, having chain lengths of from 16 to 32 carbon atoms. Petroleum based hydrocarbons having these chain lengths include mineral oil (also known as “liquid petrolatum”) and petrolatum (also known as “mineral wax,” “petroleum jelly” and “mineral jelly”). Mineral oil usually refers to less viscous mixtures of hydrocarbons having from 16 to 20 carbon atoms. Petrolatum usually refers to more viscous mixtures of hydrocarbons having from 16 to 32 carbon atoms. Petrolatum and mineral oil are particularly preferred emollients for compositions of the present invention. An exemplary use of an emollient within the context of the present invention is use in conjunction with a fibrous personal care absorbent article, such as a diaper. Further discussion of this use is found in Section III, above.

Another type of product that may be formulated from a composition of the present invention is a cream. Another type of product that may be formulated from a subject solution is a lotion. Formulations for these types of products are well known in the art.

Yet another type of product that may be formulated from a composition of the present invention is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble.

Another type of formulation is an emulsion. Emulsifiers may be nonionic, anionic or cationic and examples of emulsifiers are described in, for example, U.S. Pat. Nos. 3,755,560, and 4,421,769, incorporated herein by reference. Lotions and creams can be formulated as emulsions as well as solutions. Single emulsions for topical preparations, such as lotions and creams, of the oil-in-water type and water-in-oil type are well-known in the art. Multiphase emulsion compositions, such as the water-in-oil-in-water type, are also known, as disclosed, for example, in U.S. Pat. No. 4,254,105. Triple emulsions are also useful for topical administration of the present invention and comprise an oil-in-water-in-silicone fluid emulsion as disclosed, for example in U.S. Pat. No. 4,960,764.

Another emulsion useful in the topical compositions is a micro-emulsion system. For example, such a system comprises from about 9% to about 15% squalane, from about 25% to about 40% silicone oil; from about 8% to about 20% of a fatty alcohol; from about 15% to about 30% of polyoxyethylene sorbitan mono-fatty acid (commercially available under the trade name TWEENS) or other nonionics; and from about 7% to about 20% water.

Liposomal formulations are also useful for the compositions of the present invention. Such compositions can be prepared by combining a composition of the present invention with a phospholipid, such as dipalmitoylphosphatidyl choline, cholesterol and water according to known methods, for example, as described in Mezei et al. (1982) J. Pharm. Pharmacol. 34:473-474, or a modification thereof. Lipids suitable for forming liposomes may be substituted for the phospholipid, as may be lecithin, as well. The liposome preparation is then incorporated into one of the above topical formulations (for example, a gel or an oil-in-water emulsion) in order to produce the liposomal formulation. Other compositions and pharmaceutical uses of topically applied liposomes are described for, example, in Mezei (1985) Topics in Pharmaceutical Sciences, Breimer et al. eds., Elsevier Science, New York, N.Y., pp. 345-358.

Compounds in transdermal delivery systems are frequently attached to an skin-adhesive solid support. The compound of interest can also be combined with a penetration enhancer, e.g., Azone (1-dodecylazacycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid. Published U.S. Patent application U.S. Ser. No. 2002/0106388 describes formulations in a microencapsulated form in a slurry, which is used to impregnate body garments, such as pantyhose, for extended application to the user's skin. Such application may be advantageous in conjunction with the compositions of the present invention.

This invention also includes compositions described above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the oral compositions discussed above can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh.

Some examples of suitable excipients for oral preparations include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound may be effective over a wide dosage range and is generally administered in a pharmaceutically- or cosmetically-effective amount, as described above. It, will be understood, however, that the amount of the compound actually administered will, in the case of a pharmaceutical, be determined by a physician, in the light of the relevant circumstances, including the condition to be treated, the chosen route of administration, the actual compound administered, the age, weight, and response of the individual patient, the severity of the patient's symptoms, and the like. In the case of a cosmetic or over-the-counter skin care preparation, the actual amount of compound desired to be administered by the consumer will be recommended by the manufacturer, based on the manufacturer's test results, which may, in whole or in part, be determined on the basis of one or more of the in vitro and/or in vivo tests described herein.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices which deliver the formulation in an appropriate manner.

Other suitable carriers and their formulations are described in Remington: The Science and Practice of Pharmacy 1995, edited by E. W. Martin, Mack Publishing Company, 19th edition, Easton, Pa.

### EXAMPLES

The following preparations and examples are given to enable those skilled in the art to more clearly understand and to practice the present invention. They should not be considered as limiting the scope of the invention, but merely as being illustrative and representative thereof.

Efforts have been made to ensure accuracy with respect to numbers used (e.g., amounts, temperatures, etc.), but some experimental error and deviation should, of course, be allowed for as well as due to differences such as, for example, in calibration, rounding of numbers, and the like.

### Example 1

### Preparation of Estrogen Binding Agent-Free Soy Protein Extract

40 milliters (mL) of ethyl alcohol and 10 mL of water were added to 2 grams (g) of soymilk powder. The mixture was agitated for 4h at 22° C. on a horizontal shaker. The mixture was centrifuged and the supernatant decanted. The resulting cake was suspended in a mixture of 50 mL (80:20) ethyl alcohol/water. This suspension was also centrifuged and the supernatant decanted. The cake was then dried under vacuum resulting in 1.4 g dry powder.

Chromatographic analysis of the soy powder was carried out using High Performance Liquid Chromatography (HPLC). The results demonstrated that it is free of estrogen binding agents. More than 90% of putative estrogen binding agents, specifically flavones, isoflavones, and their glucosides were removed as compared to the original soy milk powder before extraction. Mammalian estrogen receptor binding assays also confirmed these results. Protease inhibition activity was also assessed, as described in Example 2, below, and found to be equivalent to or slightly higher than the original soy milk powder.

### Example 2

### Trypsin Inhibition Assay of Soy Protein Extract

The trypsin inhibitory activity of various extracts was determined using a commercial kit, following the protocol supplied by the vendor, Diapharma (West Chester, Ohio; www.diapharma.com). Trypsin catalyses the hydrolysis of p-nitroaniline (pNA) from a standard peptide substrate (S-2222). The reaction rate increases linearly with increasing activities of trypsin up to at least 4.8 μkat/l, which corresponds to a trypsin concentration of 2 mg/l. The rate at which pNA is released is followed on a photometer at 405 nm.

Test extracts were prepared as 1% (w/v) in deionized water and sonicated at 30° C. for 40 minutes. Samples were diluted to 0.1 and 0.01% and incubated with 25 ng of trypsin, prepared under manufacturer's instructions. Each extract was tested in duplicate, correcting for the baseline absorbance of the blank, and the percent inhibition of trypsin cleavage of the substrate by the test agents calculated.

In the extract described in Example 1, protease inhibition activity was found to be equivalent to or slightly higher than the original soy milk powder.

| | | Protease |
|---|---|---|
| Sample Description | Flavones + isoflavones (ppm) | Inhibition Activity |
| Soy Milk Powder | 1300 | 100% |
| Soy Protein Extract | <20 | 110% |

### Example 3

### Determination of Activity Utilizing the Cell Elam Assay

Endothelial-Leukocyte Adhesion Molecule (ELAM), also known as E-selectin, is expressed on the surface of endothelial cells. In this assay, lipopolysaccharide (LPS) and IL-1β are used to stimulate the expression of ELAM; test agents are tested for their abilities to reduce this expression, in accordance with studies showing that reduction of leukocyte adhesion to endothelial cell surface was associated with decreased cellular damage (e.g., Takada, M. et al., Transplantation 64: 1520-25, 1997; Steinberg, J. B. et al., J. Heart Lung Trans. 13:306-313, 1994).

Endothelial cells may be selected from any of a number of sources and cultured according to methods known in the art; including, for example, coronary artery endothelial cells, human brain microvascular endothelial cells (HBMEC; Hess, D. C. et al., Neurosci. Lett. 213(1): 37-40, 1996), or lung endothelial cells. Cells are conveniently cultured in 96-well plates, then stimulated by adding a solution to each well containing 10 micrograms (ug)/ml LPS and 100 pg/ml IL-1β for 6 hours in the presence of test agent (specific concentrations and time may be adjusted depending on the cell type). Treatment buffer is removed and replaced with pre-warmed Fixing Solution® (100 microliters (uL)/well) for 25 minutes at room temperature. Cells are then washed 3×, then incubated with Blocking Buffer (PBS+2% FBS) for 25 minutes at room temperature. Blocking Buffer containing Monoclonal E-Selectin Antibody (1:750, Sigma Catalog #S-9555) is added to each well. Plates are sealed and stored at 4° overnight. Plates are then washed 4× with 160 uL Blocking Buffer per well. Second Antibody-HRP diluted 1:5000 in Blocking Buffer is then added (100 uL/well), and plates incubated at room temperature (protected from light) for two hours. Plates are then washed 4× with Blocking Buffer before addition of 100 uL of ABTS Substrate solution at room temperature (Zymed, Catalog #00-2024). Wells are allowed to develop for 35 minutes, before measurement at 402 nm in a Fluoroskan® Reader with shake program for 10 seconds. Positive results are recorded as a decrease in ELAM concentration in tested wells, as compared to control wells.

### Example 4

### High Glutamate-Induced Oxidative Stress Assay (HGOS)

This procedure is used to induce high glutamate-induced oxidative stress (HGOS) in a dopaminergic neuronal cell line. Using this assay, the potency and efficacy of test articles against HGOS neuronal cell injury and cell death can be established in a high throughput manner.

### Materials

Dopaminergic neuronal cell lines
DMEM-No Glucose (Life Technologies Cat # 11966-025)
L-glutamine (Life Technologies Cat # 25030-081)
L-glutamic acid, monosodium salt (Sigma Cat # G5889)
D-glucose (Sigma Cat # G-6151)
10× HBSS buffer (pH 7.4) (950 ml Pyrogen-free water, 2.44 g/L MgCl2.6H20, 3.73 g/L KCl, 59.58 g/L Hepes, 58.44 g/L NaCl, 1.36 g/L KH2PO4, 1.91 g/L CaCl2 .2H2O and pH to 4.5 with HCl)
Cell Tracker Green fluorescent dye (Molecular Probes, Cat # 2925). Prepare a 5 μM solution in pre-warmed HBSS just prior to use.
Sterile 96-well plates precoated with poly-D-lysine (Corning Catalog # 3665)
96-well deep well mother plate, DyNA Block 1000 (VWR Catalog # 40002-008)

### Neuronal Cells

The cells are seeded into 96-well plates at a density of 2000 per well and left to grow for 72 hours in a 33° C. incubator with 5% CO₂ in air atmosphere. The passage number of the cells for each assay experiment is no later than p11 in order to minimize experimental variation.

### Compound Preparation in Deep-well Mother Plates

VWRBrand DyNA Block 1000, deep well mother plates (VWR Cat. # 40002-008) are used for the preparation of the test compounds.

All compounds are dissolved in DMEM-No Glu containing 1 mM glucose, 30 mM glutamate and 1× Pen/Strep. DMEM-No Glu with 1 mM glucose and 1× P/S is used as the negative control, DMEM-No Glucose with 1mM glucose, 100 M glutamate is used as a positive control and 100 μM Glutathione is added to the positive control as a standard. All of the procedures for this involving the making and dilution of compounds are performed using aseptic conditions and with minimal light.

### Cell Preparation

The plates are removed from the incubator and examined under the microscope for morphological appearance and density. Using an aseptic technique and an 8-channel aspirator the media is carefully removed from the cells and replaced with 200 μl of 1×HBSS. This is done as quickly as possible to prevent the cells drying out. The plates are then placed in the humidified 37° C. incubators of the Biomek 2000 Side Loader. Four plates are washed at a time so as to minimize the time that the cells are sitting in 1× HBSS prior to addition of the compound test solution.

### Experimental Setup

The Beckman Biomek workstations are used to load the compounds and controls from the mother plates onto the cell plates that are prewashed with HBSS under sterile conditions. The plates are incubated in the upper HTS incubator at 37° C. in 5% CO2 for exactly 16 hrs. The following day, using the Beckman Biomek workstations, the plates are removed from the incubator. Using Cell Tracker Addition, the compounds are removed from the plates, washed once with 200 μM of pre-warmed 1× HBSS and then 100 μL of 5 μM Cell Tracker Green is added to each well. The plates are incubated at 37° C. for 30 min to allow the dye to enter the cell and be cleaved by the esterases. After washing the cells twice with prewarmed 1×HBSS, the plates are read with the 485 excitation; 538 emission filter pair on a Fluoroskan.

### Example 5

### Mammalian Estrogen Receptor (ER) Binding Activity

ER binding activities of soy product and extracts made in accordance with Example 1 were tested for activity by competition for binding of radioligand to two forms of recombinant human estrogen receptors, ER-alpha and ER-beta, using standard methods (Obourn, et al, Biochem. 32: 6229-6236, 1993), with 0.5 nM tritiated estradiol as radioligand and diethylstilbestrol as standard, on receptors from human recombinant insect Sf9 cells.

IC₅₀ values were determined by non-linear, least squares regression analysis using Data Analysis Toolbox™ (MDL Information Systems, San Leandro, Calif., USA). Ki values were calculated using the Cheng and Prusoff equation (Cheng, et al., Biochem. Pharmacol. 22:3099-3108, 1973).

In assays performed as described above, native soy product exhibited IC₅₀s of 14 and 0.6 micrograms/liter, against ER-alpha and ER-beta (Kᵢ's, 4.02 and 0.14 micrograms/l); soy extracts did not exhibit statistically measurable IC₅₀s (maximum inhibition of binding of ER-alpha of approximately 31% at a concentration of 1000 micrograms/liter). This demonstrates that the extracts exhibited estrogen receptor binding activity less than about 2% of the source material.

### Example 6

### Healthy Skin Assessment/Improvement of Skin Appearance

A double blind placebo-controlled clinical study is conducted on human female subjects ages 21-40 to assess the efficacy of compositions to affect tone and tactile properties of human skin. Subjects are directed to apply formulation or carrier-matched placebo formulation to ½ of the face daily for 6 weeks. Scaling, moisturization, oiliness, smoothness, redness, blotchiness are recorded by instrumental measurements and digital photography, and by self-assessment.

### Example 7

### Photoprotection Activity

Female C3H/HeNTac mice (Taconic, Germantown, N.Y.) are shaved (dorsal back areas only) and administered 50 mg each of formulations of the invention mixed in neutral cream vehicle in 1% or 5% (w/w) dispersions. Ultraviolet (UV) lights having defined emission characteristics (80% UVB, 4% UVA, remainder visible; Westinghouse FS20 lamps) are used as UV source to expose animals. Lamps are mounted 20 cm above the mouse cage bottom, and mice are irradiated for 60 minutes (3.6-3.7 m²/s radiation). Mice are then analyzed for skin redness and epidermis is further analyzed for in situ formation of pyrimidine cyclobutane dimmers, according to methods known in the art (McVean, et al., Mol. Carcinog. 24: 169-176, 1999). Inhibition of dimmer formation is evidence of photoprotective activity.

### Example 8

### Depigmentation Assay

A skin lightening assay is commercially available (MatTek Corporation, Ashland, Mass.; “MelanoDerm” assay; www.mattek.com). The system used consists of normal, human-derived epidermal keratinocytes (NHEK) and melanocytes (NHM) which have been cultured to form a multilayered, highly differentiated model of the human epidermis. The tissues are produced using serum free medium without artificial stimulators of melanogenesis and maintained with regular applications of “maintenance medium”. The cultures are grown on cell culture inserts at the air-liquid interface, allowing for topical application of skin lighteners or self-tanning agents. NHM localized in the basal cell layer of MelanoDerm are dendritic and spontaneously produce melanin granules which progressively populate the layers of tissue. The topical application of known inhibitors of melanogenesis significantly reduce melanin production and macroscopic darkening. Conversely, NHM within the tissue will respond to known stimulants of melanogenesis. The organotypic cultures allow for topical or subcutaneous application of melanogenesis inhibitors or stimulators. Test agents are applied on a daily basis or using any other dosing schedule as required. Tissues can be analyzed visually or microscopically on a daily basis or as required. Micrographs (4× and 10×) are scored (on an arbitrary scale of −3 to +3) for pigmentation (number of pigmented melanocytes and degree of pigmentation) and dendricity (an indication of viability) by a blinded scorer. At the end of the experiment the tissues are processed and stained (H&E) for histological analysis. Cross-sectioned histological samples are evaluated for cell morphology and normal skin cell layering, toxicity and overall appearance. Samples are scored for histological improvement or damage according to standard scoring criteria.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

1. A skin care composition, comprising
a plant protein extract supplemented with at least 1% (w/w) of a non-alpha tocopherol or a non-alpha tocopherol enriched tocopherol composition.

2. The skin care composition of claim 1, wherein said non-alpha tocopherol is beta tocopherol.

3. The skin care composition of claim 1, wherein said non-alpha tocopherol is delta tocopherol.

4. The skin care composition of claim 1, wherein said non-alpha tocopherol is gamma tocopherol.

5. The skin care composition of claim 1, wherein said plant protein extract is a soy protein extract.

6. The skin care composition of claim 5, wherein said non-alpha tocopherol is beta tocopherol.

7. The skin care composition of claim 5, wherein said non-alpha tocopherol is delta tocopherol.

8. The skin care composition of claim 5, wherein said non-alpha tocopherol is gamma tocopherol.

9. The skin care composition of claim 5, wherein said soy protein extract retains protein function, as evidenced by at least about 80% of the trypsin inhibitory activity present in the starting material from which the soy protein extract is produced.

10. The skin care composition of claim 5, wherein said soy protein extract is essentially depleted of mammalian estrogen receptor binding activity.

11. The skin care composition of claim 10, wherein said soy protein extract contains less than about 10% of the mammalian estrogen receptor binding activity present in the soy material from which the extract is generated.

12. The skin care composition of claim 10, wherein said soy protein extract contains less than about 5% of the mammalian estrogen receptor binding activity present in the soy material from which the extract is generated.

13. The skin care composition of claim 10, wherein said soy protein extract contains less than about 1% of the mammalian estrogen receptor binding activity present in the soy material from which the extract is generated.

14. The skin care composition of claim 5, which further includes a retinoid.

15. The composition of claim 14, wherein said retinoid is retinoic acid.

16. The skin care composition of claim 5, formulated for topical administration to the skin of a mammalian subject.

17. The skin care composition of claim 16, wherein said composition is included in a topical cosmetic formulation.

18. The skin care composition of claim 16, wherein said composition is included in a topical pharmaceutical formulation.

19. The skin care composition of claim 5, wherein said composition is formulated for inclusion in a body waste containment article.

20. The skin care composition of claim 5, formulated for transdermal administration to a mammalian subject.

21. The skin care composition of claim 5, formulated for oral administration to a mammalian subject.

22. The skin care composition of claim 21, wherein said composition is formulated as a nutritional cosmetic formulation.

23. The skin care composition of claim 1, wherein said plant protein extract is a colloidal oatmeal preparation.

24. The skin care composition of claim 23, wherein said colloidal oatmeal preparation is essentially depleted of estrogen receptor binding activity.

25. The skin care composition of claim 24, wherein said colloidal oatmeal preparation contains less than about 5% of the mammalian estrogen receptor binding activity present in the soy material from which the extract is generated.

26. The skin care composition of claim 24, wherein said colloidal oatmeal preparation contains less than about 2% of the mammalian estrogen receptor binding activity present in the soy material from which the extract is generated.

27. The skin care composition of claim 24, wherein said colloidal oatmeal preparation contains less than about 1% of the mammalian estrogen receptor binding activity present in the soy material from which the extract is generated.

28. The skin care composition of claim 23, wherein said non-alpha tocopherol is delta-tocopherol.

29. The skin care composition of claim 23, wherein said non-alpha tocopherol is beta-tocopherol.

30. The skin care composition of claim 23, wherein said non-alpha tocopherol is gamma-tocopherol.

31. The skin care composition of claim 23, wherein said composition is formulated for topical delivery to the skin.

32. The skin care composition of claim 31, formulated for inclusion in a body waste containment article.

33. The skin care composition of claim 23, wherein said composition is formulated for oral delivery.

34. A baby skin care composition, comprising a plant protein extract supplemented with at least 1% (w/w) of a non-alpha tocopherol or a non-alpha tocopherol enriched tocopherol composition.

35. The baby skin care composition of claim 34, wherein said plant protein extract is a soy protein extract.

36. The baby skin care composition of claim 34, wherein said plant protein extract is a colloidal oatmeal preparation.

37. The baby skin care composition of claim 34, wherein said non-alpha tocopherol is beta tocopherol.

38. The baby skin care composition of claim 34, wherein said non-alpha tocopherol is delta tocopherol.

39. The baby skin care composition of claim 34, wherein said non-alpha tocopherol is gamma tocopherol.

40. The baby skin care composition of claim 35, wherein said soy protein extract retains protein function, as evidenced by at least about 80% of the trypsin inhibitory activity present in the starting material from which the soy protein extract is produced.

41. The baby skin care composition of claim 35, wherein said soy protein extract is essentially depleted of mammalian estrogen receptor binding activity.

42. The baby skin care composition of claim 35, wherein said soy protein extract contains less than about 5% of the mammalian estrogen receptor binding activity present in the soy material from which the extract is generated.

43. The baby skin care composition of claim 35, wherein said soy protein extract contains less than about 2% of the mammalian estrogen receptor binding activity present in the soy material from which the extract is generated.

44. The baby skin care composition of claim 35, wherein said soy protein extract contains less than about 1% of the mammalian estrogen receptor binding activity present in the soy material from which the extract is generated.

45. The baby skin care composition of claim 34, formulated for topical administration to the skin of a mammalian baby.

46. The baby skin care composition of claim 45, wherein said composition is formulated for inclusion in a body waste containment article.

47. The baby skin care composition of claim 34, wherein said composition is included in a topical pharmaceutical formulation.

48. The baby skin care composition of claim 34, formulated for transdermal administration to a mammalian baby.

49. The baby skin care composition of claim 34, formulated for oral administration to a mammalian baby.

50. A method of treating or ameliorating the symptoms of an inflammatory skin condition in a mammalian subject, comprising
administering to the subject a cosmetically or pharmaceutically effective amount of a composition according to claim 1.

51. The method of claim 50, wherein the inflammatory skin condition is retinoid-induced skin irritation.

52. The method of claim 50, wherein the inflammatory skin condition is sunburn.

53. The method of claim 50, wherein the inflammatory skin condition is diaper rash.

54. A skincare kit, comprising
a composition according to claim 1, and
instructions for applying such composition to the skin of a mammalian subject.
